(19) United States
(12) Patent Application Publication
(10) Pub. No.: US 2008/0004440 A1
(43) Pub. Date: Jan. 3, 2008

(54) SCAR TREATMENT USING PROTEIN PHOSPHATASE INHIBITORS

(75) Inventors: Lizhen Zhao, Nanjing (CN); Yan Gao, Providence, RI (US)

Correspondence Address: SEED INTELLECTUAL PROPERTY LAW GROUP PLLC, 701 FIFTH AVE, SUITE 5400, SEATTLE, WA, 98104, US

(73) Assignee: BIOBOTANIC CORP., East Providence, RI (US)

(21) Appl. No.: 11/765,872

(22) Filed: Jun. 20, 2007

Related U.S. Application Data
(60) Provisional application No. 60/815,406, filed on Jun. 20, 2006.

(51) Int. Cl. C07D 307/93; C07D 245/02; C07D 315/00; C07D 263/32

(52) U.S. Cl. 540/460; 548/236; 549/300; 549/415; 549/420

(57) ABSTRACT

A method for treating a scar comprises administering to a scar a pharmaceutically effective amount of one or more blistering agents in a pharmaceutically acceptable carrier.

### CROSS-REFERENCE TO RELATED APPLICATION

This application claims the benefit under 35 U.S.C. §119(e) of U.S. Provisional Patent Application No. 60/815,406 filed Jun. 20, 2006, which application is incorporated herein by reference in its entirety.

### BACKGROUND

1. Technical Field

This invention is related to treating scars, in particular, to treating scars by causing blistering and necrosis of scar tissue.

2. Description of the Related Art

Hypertrophic scars and keloids can be described as variations of typical wound healing. Typically, when a wound heals, anabolic and catabolic processes achieve an equilibrium approximately 6-8 weeks after an initial injury. At this stage, the strength of the wound is approximately 30-40% of that of healthy skin. As the scar matures, the tensile strength of the scar improves as a result of progressive cross-linking of collagen fibers. At this point, the scar is usually hyperemic and may be thickened, but it tends to subside gradually over months. A fully mature scar is inconspicuous because it is typically flat, white, pliable, and possibly stretched.

When an imbalance occurs between the anabolic and catabolic phases during the healing process, more collagen is produced than is degraded, and the scar grows in all directions. The scar is elevated above the skin and remains hyperemic. This excessive scar tissue is classified as either a keloid or a hypertrophic scar. They differ from healthy skin by a rich vasculature, high mesenchymal cell density, and thickened epidermal cell layer. In both keloid and hypertrophic scars, collagen nodule is indicated as the identifying structural unit. The nodule, which is absent from mature scars, contains a high density of fibroblasts and unidirectional collagen fibrils in a highly organized and distinct orientation. The difference between a keloid and a hypertrophied scar is that a keloid continues to enlarge beyond the original size and shape of the wound, while a hypertrophied scar enlarges within the confines of the original wound. The most consistent histologic difference is the presence of broad, dull, pink bundles of collagen in keloids, which are not present in hypertrophic scars.

Scars can be treated by non-surgical and surgical means. Non-surgical treatments include occlusive dressings (e.g., silicone gel sheets and dressings, non-silicone occlusive sheets, Cordran® tape and Scarguard®); compression therapies involving pressure; and corticosteroid therapies to reduce collagen synthesis (specifically, intralesional corticosteroid injections). Surgical treatments include cryotherapy, laser, surgical excision and the like. These surgical treatments typically show scar recurrences from 25% to 100%, and thus are not efficient.

New treatments for keloids and hypertrophic scars include injections of intralesional interferon, verapamil, bleomycin, 5-fluorouracil (5-FU), retinoic acid, imiquimod, tacrolimus, or botulinum toxin. Interferon has demonstrated ability to reduce fibroblast production of collagen. Verapamil is a calcium channel blocker that blocks the synthesis/secretion of extracellular matrix molecules and increases fibrinase. Bleomycin injections cause necrosis of keratinocytes with a mixed inflammatory infiltrate. 5-FU inhibits fibroblastic proliferation in tissue culture.

Current regimens for scar treatments are limited by low efficacy, high recurrence rates and side effects caused by drug therapies. There remains a need in the art for effective scar treatments.

### BRIEF SUMMARY

One embodiment describes a method for treating a scar, the method comprising administering to the scar a pharmaceutically effective amount of a blistering agent in a pharmaceutically acceptable carrier.

More specifically, the blistering agent is a compound of Formula (I): 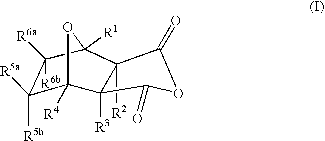
wherein R¹, R², R³, R⁴, R^{5a}, R^{5b}, R^{6a} and R^{6b} are the same or different and independently, hydrogen, alkyl, alkenyl, alkynyl, alkoxy, aryl, aralkyl, acyl, halogen, CN or NO₂, or a stereoisomer, acid or salt thereof.

In other embodiments, the blistering agent is a protein phosphatase inhibitor.

### DETAILED DESCRIPTION

In certain embodiments, the present invention provides a method for treating scars through modulating cell permeability. More specifically, the method employs a blistering agent to alter cell permeability, thereby causing blistering of a hypertrophic or keloid scar. The blistering leads to necrosis of the scar, which can then be removed through debridement. The removal of the necrotic scar tissue allows the body's natural healing mechanisms to stimulate new tissue growth. Optionally, an additional wound healing agent can be used in a combination therapy to promote healing after the debridement.

Before describing the present invention in detail, it is to be understood that this invention is not limited to specific pharmacologically active protein phosphates inhibitors, carriers, formulation types, treatment regimens, and so forth, as such may vary. It is also to be understood that the terminology used herein is for the purpose of describing particular embodiments only, and is not intended to be limiting.

It must be noted that, as used in this specification and the appended claims, the singular forms “a”, “an” and “the” include plural referents unless the context clearly dictates otherwise.

“Pharmaceutically acceptable”, such as in the recitation of a “pharmaceutically acceptable carrier,” or a “pharmaceutically acceptable derivative”, means a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained.

The terms “treating” and “treatment” as used herein refer to actions that reduce the severity and/or frequency of symptoms, eliminate symptoms and/or their underlying cause, prevent the occurrence of symptoms and/or their underlying cause, and improve or remediate damage. The present method of “treating” a mammal, including a human patient, as the term is used herein, thus encompasses both prevention of scars in a predisposed individual and treatment, including removal of scars in a clinically symptomatic individual.

“Blistering agent” is an agent of chemical or biologic origin, which, when used in a well-controlled manner, causes localized blisters upon contact with a scar. Blisters refer to localized fluid accumulation under the scar.

As used herein, a blistering agent typically increases cell permeability, which consequently leads to extracellular fluid accumulation. In addition, increased endothelial permeability increases volume of plasma and serum permeated out of the capillaries. When an increasing amount of extracellular fluid accumulates and reaches a surface of the scar, blisters develop.

The change in permeability can directly cause necrosis of cells. Moreover, where there is endothelial damage, increased plasma and serum losses induce local coagulation and a sharp increase of platelets. As a result, capillary blood flow is clogged, thereby cutting off the nutrient supply network to the local tissue and eventually causing local viable tissue to necrotize.

When applied either topically or intralesionally, the blistering agent can be absorbed by a local capillary network and diffuses along the same network. The blistering agent increases the network's endothelial permeability and causes damage to the tissue pertaining to the network.

By an “effective” amount or a “therapeutically effective amount” of a pharmacologically active agent is meant a nontoxic but sufficient amount of the drug or agent to provide the desired effect, i.e., prevention or treatment of scars. The amount that is “effective” will vary from subject to subject, depending on the size of lesion, age and general condition of the individual, mode of administration, and the like. Thus, it is not always possible to specify an exact “effective amount.” However, an appropriate “effective” amount in any individual case may be determined by one of ordinary skill in the art using routine experimentation.

The term “topical application” is used in its conventional sense to mean delivery of a topical drug or pharmacologically active agent to the skin or mucosal tissue, as in, for example, the treatment of warts.

The term “intralesional injection” means injecting a drug directly into the skin lesion to let the drug pass a barrier zone and establish a sub-epidermal depot, thus allowing a higher concentration of the drug to act at the site of the lesion.

The term “pharmaceutically acceptable carrier” or “carrier” refers to any inert material (e.g., a diluent), organic or inorganic, that does not cause significant irritation to a subject and does not abrogate the biological activity and properties of an administered active ingredient. Suitable carriers can be selected based on the means of administration, as is understood by one skilled in the art. Exemplary carriers suitable for topical applications include, but are not limited to: water, alcohol, acetone, an ointment, gel spray, or paste. Exemplary carriers suitable for intralesional injection include, but are not limited to: saline, water, alcohol, acetone, and oil.

The term “scar” includes all types of scar tissue formation after an injury to the skin that causes an original skin wound. In general, scars include hypertrophic scars, keloid scars, contracture scars, acne scars and hypotrophic scars. Hypertrophic scars grow above the epidermal layer of skin; keloid scars grow above epidermal layer and beyond the limit of the original skin wound; hypotrophic scars are below the epidermal level of surrounding skin.

A “scar wound” is a lesion where scar tissue has necrotized and/or is removed as part of a treatment of the scar. The scar wound is not to be confused with an original skin wound, the latter being caused by an injury. The scar wound is typically surrounded by viable skin tissue, which is capable of wound healing and re-growth through the body's natural healing mechanism. When a scar wound is caused by a blistering agent, the surrounding viable skin tissue may or may not be damaged at the same time. Preferably, the surrounding viable skin tissue is not damaged.

In one embodiment, a method for treating scar or scar tissue is described, comprising applying a blistering agent to the scar tissue, causing blistering and necrosis of the scar tissue, and removing the scar tissue.

The blistering agent can be applied topically, or by intralesional injection. The blistering agent can cause local tissue damage and necrotize the scar tissue. Preferably, the above process stimulates the body's own wound healing processes.

As discussed herein, once the blistering agent permeates into the capillary blood stream, it diffuses along the local capillary network. The blistering agent increases local cell permeability, especially endothelial permeability, and causes local edema and blistering of the scar surface. Eventually, the administration of the blistering agent will cause necrosis in the scar tissue matrix.

Depending on the type and dosage of the blistering agent, the method of administration, condition of a patient, as well as nature of a scar, scar tissue takes time, from minutes to days, to necrotize. After the scar tissue has necrotized, debridement (i.e., removal of the necrotic tissue) can be achieved by mechanical methods, or by chemical and biological debridement agents. Mechanical methods include physical peeling and surgical excision. Excision can be more selective if the necrotic tissue is marked by Masson staining or Vimentin immuno-histochemical staining. In the event of chemical debridement, agents such as Elase (fibrinolysin and desoxyribonuclease by Warner-Lambert, Parker-Davis) can be used to assist. Certain burn treatment drugs, such as Lyzenic (BioBotanic Corp.), have both debridement and healing functions. Detailed description of Lyzenic can be found in U.S. Provisional Application No. 60/801,971, in the name of BioBotanic Corp., the assignee of the present application.

After the necrotic tissue is removed, or “debrided”, the mammalian body's spontaneous wound healing ability will heal the scar wound area by stimulating new skin growth. This healing process can be promoted by proper treatments, especially wound treatments that provide antibiotics, analgesics, growth factors and moist environment, etc. If the scar wound depth is within the dermal layer of the skin, the wound can spontaneously heal without scarring. If the wound depth is below the dermal layer of human skin, the wound can be recovered without scarring with assistance of wound treatments.

With the latest development in wound treatment, such as moist environment therapy and growth factors, humans have become more and more confident in treating skin wounds faster and without scarring. It has been widely reported that superficial burn wounds, the wounds that damage epidermal and dermal layers of human skin, can be recovered without scarring. In some cases, even a full thickness burn (third degree burn) can be treated without scarring. For example, the herbal medicine Lyzenic can treat deep second and third degree burns without scarring.

These effective wound treatments can form part of a combination therapy in scar treatment by treating a scar wound caused by the application of one or more blistering agents. Thus, in a further embodiment, a method of treating scars comprises: applying one or more blistering agents to a scar, allowing the scar to blister and necrotize to form a scar wound, and treating the scar wound.

If necessary, once a scar wound, including the scar and surrounding skin area, has healed satisfactorily and the skin returns to its normal tension, the claimed treatments can be repeated to further treat the scar. This cycle can be repeated multiple times until all the scar tissue is removed.

Examples of Blistering Agents:

The pharmacologically active base of the invention is one or more blistering agents. The blistering agent can be, for example, a cantharidin derivative or a protein phosphatase inhibitor.

In one embodiment, the blistering agent is a cantharidin derivative, represented by the following chemical formula (I): 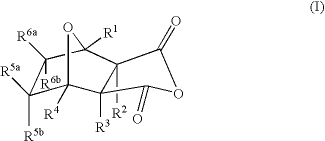
wherein R¹, R², R³, R⁴, R^{5a}, R^{5b}, R^{6a} and R^{6b} are the same or different and independently, hydrogen, alkyl, alkenyl, alkynyl, alkoxy, aryl, aralkyl, acyl, halogen, CN or NO₂, or an stereoisomer or salt thereof.

As used herein, alkyl refers to an optionally substituted monovalent saturated hydrocarbon structure, in which the carbons are arranged in either a linear or branched manner. Lower alkyl refers to alkyl groups of 1 to 5 carbon atoms. Examples of lower alkyl groups include methyl, ethyl, propyl, isopropyl, butyl, s- and t-butyl and the like. When an alkyl residue having a specific number of carbons is named, all geometric isomers having that number of carbons are contemplated; thus, for example, “butyl” is meant to include n-butyl, sec-butyl, isobutyl and t-butyl; propyl includes n-propyl and isopropyl.

“Alkenyl” refers to an optionally substituted monovalent hydrocarbon structure of between 2 and 20 carbon atoms with at least one double bond. Examples include without limitation: ethenyl, propenyl, butenyl, pentenyl, hexenyl, butadienyl, pentadienyl and the like. Unless specified otherwise, the alkyl can be optionally substituted with a halogen (F, Br, Cl or I), alkoxy, amine or the like.

“Alkynyl” refers to an optionally substituted monovalent hydrocarbon structure of between 2 and 20 carbon atoms with at least one triple bond. Examples include without limitation: ethynyl, propynyl, butynyl, pentynyl, hexynyl, methylpropynyl, 4-methyl-1-butynyl, 4-propyl-2-pentynyl and the like.

“Alkoxy” refers to an optionally substituted radical of the formula —O-alkyl. Examples include methoxy, ethoxy, propoxy, isopropoxy, and the like. Lower-alkoxy refers to groups containing one to five carbons.

“Aryl” refers to optionally substituted phenyl or naphthyl. Exemplary substituents for aryl include one or more of halogen, hydroxy, alkoxy, amino, mercapto or the like.

“Aryloxy” refers to phenoxy or naphthoxy.

“Aralkyl” refers to an alkyl residue substituted with at least an aryl group, as defined herein. The aralkyl can be typically represented by the formula aryl-alkyl-. Exemplary aralkyls include without limitation phenylmethyl (i.e., benzyl) or phenylethyl group.

“Acyl” refers to a radical of the formula —C(═O)—R, wherein R is alkyl, alkenyl, alkynyl, aryl, alkoxy, aryloxy, heterocycle or heteroaryl, where alkyl, alkenyl, alkynyl, aryl, alkoxy, aryloxy, heterocycle and heteroaryl are as defined herein. Representative acyl groups include acetyl, benzoyl, propionyl, isobutyryl, t-butoxycarbonyl, and the like.

Halogen refers to F, Cl, Br or I.

“Substituted” means any of the above groups (e.g., alkyl, alkoxy, acyl, aryl,) wherein at least one hydrogen atom is replaced with a substituent. In the case of an oxo substituent (“═O”), two hydrogen atoms are replaced. Substituents include halogen, hydroxy, oxo, alkyl, aryl, alkoxy, aryloxy, acyl, mercapto, cyano, alkylthio, arylthio, —NRₐR_{b}, —NRₐC(═O)R_{b}, —NR_{C}C(═O)NRₐR_{b}, —NRₐC(═O)OR_{b}, —NRₐSO₂R_{b}, —C(═O)NRₐR_{b}, —OC(═O)Rₐ, —OC(═O)ORₐ, —OC(═O)NRₐR_{b}, —NRₐSO₂R_{b} or a radical of the formula —Y-Z-Rₐ where Y is alkanediyl, substituted alkanediyl or a direct bond, alkanediyl refers to a divalent alkyl with two hydrogen atoms taken from the same or different carbon atoms, Z is —O—, —S—, —S(═O)—, —S(═O)₂—, —N(R_{b})—, —C(═O)—, —C(═O)O—, —OC(═O)—, —N(R_{b})C(═O)—, —C(═O)N(R_{b})— or a direct bond, wherein Rₐ, R_{b} and R_{c} are the same or different and independently hydrogen, amino, alkyl, substituted alkyl (including halogenated alkyl), aryl, substituted aryl or aralkyl (e.g., phenylmethyl).

In one embodiment, the blistering agent is cantharidin (R²═R³═CH₃, R¹═R⁴═R^{5a}═R^{5b}═R^{6a}═R^{6b}═H):

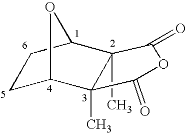

It has been indicated that cantharidin can increase endothelial permeability by attenuating phosphorylation of certain cytoskeletal proteins that could be involved in cytoskeletal rearrangement.

Cantharidin (2,3-dimethyl-7-oxabicyclo[2,2,1]heptane-2,3-dicarboxylic anhydride) was first isolated in 1810 by Robiquet. See, e.g., Robiquet, M. Ann. Chim. (1810), 76, 302-307. Its molecular formula is C₁₀H₁₂O₄ with melting point of 218° C. Cantharidin can be prepared by the methodologies as described in, e.g., Stork et al., J. Am. Chem. Soc. (1951), 73, 4501; Stork, et al., J Am. Chem. Soc. (1953), 75, 384; Schenk, et al., Naturwissenshaften (1953), 40, 531, Dauben et al., J. Am. Chem. Soc. (1980), 102, 6893-6894, and Dauben et al., J. Am. Chem. Soc. (1976), 98, 1992-1993.

Alternatively, cantharidin can be extracted from natural sources. Cantharidin is naturally produced by at least two insect-families: the Meloidae and the Oedemeridae. The first family comprises more than a thousand species, in which the Epdicauta are the most widespread. In Chinese medicine, Mylabris phalerata Pallas and Mylabris cichorii Linnaeus are the two most common sources from which cantharidin can be extracted.

In another embodiment, the blistering agent is a cantharidin analog, such as norcantharidin (7-oxabicyclo[2.2.1]heptane-2,3-dicarboxylic anhydride).

In another embodiment, the blistering agent is palasonin. This molecule is produced by a tree butea frondosa that grows in the Himalaya. Unlike cantharidin, palasonin is chiral and exists in two enantiomeric forms (represented below). The natural compound is (−)-palasonin.

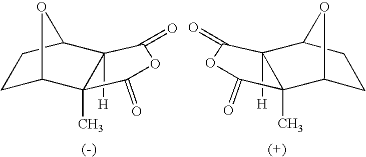

When applied to mammalian skin, a compound of Formula (I), e.g., cantharidin, can form blisters on the skin surface. Cantharidin has been used clinically to treat topical viral diseases such as herpes infection and warts. In clinical settings, for example, cantharidin (0.9% solution of collodian and acetone) has been used to treat molluscum contagiosum virus.

In accordance with one embodiment, the blistering agent can be applied carefully and sparingly to the dome of a scar with or without occlusion and left in place for at least 4 hours before being washed off. Cantharidin can cause severe blistering and lesions. It should be tested on an individual scar before treating large numbers of scars. When tolerated, this treatment can be repeated every week until the lesions clear. Usually 1-3 treatments are necessary.

In certain embodiments, the blistering agent is a protein phosphatase inhibitor. The term “protein phosphatase (PP) inhibitor” refers to an agent that inhibits biochemical functions of all types of phosphatases. In particular, it refers to PP2A inhibitors that are capable of altering cell permeability, e.g., endothelial cell permeability.

Protein phosphatases (PP) remove phosphate groups at specific serine, threonine and tyrosine sites. They play an integral role in the control of cellular phosphoproteins by mediating signal transduction events, including signal amplification, the timing of physiological responses and cross-talk between distinct signal transduction pathways. They are classified into three major groups depending on their substrate specificity and dependence on metal ions: PP1, PP2A and PP2B. The catalytic subunit of PP1 is bound to the regulatory subunits that determine the subcellular localization and activity of the enzyme. PP2A is inactivated by transient phosphorylation of tyrosine residues on the molecule. PP2B depends on the Ca²⁺-calmodulin complex for complete activation.

In various embodiments, the Kᵢ of the protein phosphatase (e.g., PP2A) inhibitor is less than 1 μM, more preferably, less than 500 nm, less than 100 nm, less than 10 nm, or less than 1 nm. This group of agents includes, but is not limited to: calyculin A; endothall; fostriecin; microcystin-LF; microcystin-LW; microcystin-RR; α-naphthyl acid phosphate; okadaic acid; ammonium okadaic salt; potassium okadaic salt; sodium okadaic salt; protein phosphatase 2A inhibitor I₁^{PP2A}, protein phosphatase 2A inhibitor I₂^{PP2A} (see, also, table 1.)

TABLE 1

| | MW | Properties |
|---|---|---|
| Calyculin A | 1009.2 | Cell-permeable. Phosphorylated polyketide. PP2A ˜ PP1 >> PP2B |
| | | (IC₅₀ for PP2A = 0.5-1.0 nM and for |
| | | PP1 = 2.0 nM). |
| Endothall | 186.2 | A specific inhibitor of PP2A (IC₅₀ = 90 nM). |
| Fostriecin | 452.4 | A potent PP2A inhibitor (IC₅₀ = 3.2 nM) Inhibits PP1 |
| | | only at higher concentrations (IC₅₀ = 131 μM). |
| Microcystin-LR | 995.2 | Cyclic peptide. PP2A ˜ PP1 >> PP2B (IC₅₀ for PP2A = 40 pM |
| | | and for PP1 = 1.7 nM). |
| Microcystin-LW | 1025.2 | A more cell-permeable analog of Microcystin-LR |
| Microcystin-RR | 1038.2 | Cyclic peptide. PP2A ˜ PP1 >> PP2B (IC₅₀ = 1.4 nM |
| | | for PP2A). |
| α-Naphthyl acid | 257.1 | Broad-spectrum protein phosphatase inhibitor. |
| Phosphate | | |
| Okadaic acid | 805.0 | Cell-permeable. PP2A > PP1 >> PP2B (IC₅₀ for PP2A = 100 pM; |
| | | for PP1 = 10-15 nM; and for PP2B = 5 μM). |
| Okadaic acid, | 822.0 | Cell-permeable. PP2A > PP1 >> PP2B. (IC₅₀ for |
| ammonium salt | | PP2A = 100 pM; for PP1 = 10-15 nM; and for PP2B = 5 μM). |
| Okadaic acid, | 843.1 | Cell-permeable. PP2A > PP1 >> PP2B. (IC₅₀ for |
| potassium salt | | PP2A = 100 pM; for PP1 = 10-15 nM; and for PP2B = 5 μM). |
| Okadaic acid, sodium | 827.0 | Cell-permeable. PP2A > PP1 >> PP2B. (IC₅₀ for |
| salt | | PP2A = 100 pM; for PP1 = 10-15 nM; and for PP2B = 5 μM). |
| Protein phosphatase | 3000 | Potently inhibits all forms of PP2A (Kᵢ ˜ 0.1 nM). |
| 2A inhibitor I₁^{PP2A}, | | |
| Protein phosphatase | 39,000 | Potently inhibits PP2A (Kᵢ ˜ 0.100 pM) |
| 2A inhibitor I₂^{PP2A}, | | |

In a particular embodiment, the blistering agent is okadaic acid or a salt thereof. Okadaic acid in nature is produced by marine algae. Okadaic Acid structure is as below (Formula II); its salt derivatives, such as okadaic sodium, potassium, ammonium and the like, when hydrolyzed, possess the same medicinal function as okadaic acid.

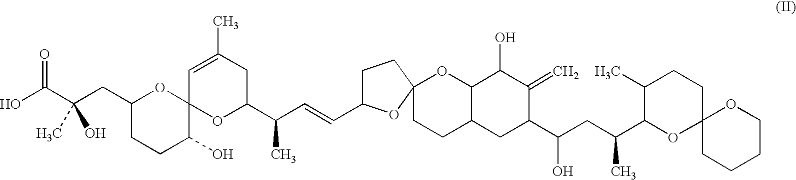

### EXAMPLE I

### Treating Scars using a Cantharidin Extract

Cantharidin was extracted from Mylabris phalerata Pallas using ethanol as an extracting medium. An ethanol solution of cantharidin at a concentration of 0.1%-2% by weight was used to treat hypertrophic and keloid scars. The formula is topically applied to a scar site with a cotton swab, approximately 0.1-2 mg per 100 mm². After about four hours, the scar site and its surrounding skin start to inflame and blister. An “artificial” scar wound similar to a second degree burn is formed. The blisters will be drained by using a surgical needle. A wound treatment drug, Lyzenic by BioBotanic Corp, was then applied to the scar wound site with gauze dressing and bandages. Within the first 24 hours, necrotic slough will be removed with dressing changes. The scar wound of this depth can usually heal in 4-7 days. Actual treating times may vary depending on the size, depth, age and location of the scar. Once the wound has healed, the original scar tissue's size and thickness should be reduced, or the scar should be totally removed with new skin growth. If necessary, the treatment can be repeated after the new skin recovers to its normal tension and flexibility.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating a scar comprising administering to the scar a pharmaceutically effective amount of a blistering agent in a pharmaceutically acceptable carrier.

2. The method for treating scars according to claim 1, wherein the blistering agent is a compound of Formula (I): 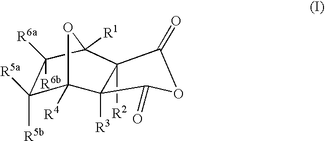
wherein R¹, R², R³, R⁴, R^{5a}, R^{5b}, R^{6a} and R^{6b} are the same or different and independently, hydrogen, alkyl, alkenyl, alkynyl, alkoxy, aryl, aralkyl, acyl, halogen, CN or NO₂, or an stereoisomer, acid or salt thereof.

3. The method of claim 2 wherein the blistering agent cantharidin.

4. The method of claim 2 wherein the blistering agent is cantharidic acid, norcantharidin or palasonin.

5. The method for treating scars according to claim 1, wherein the blistering agent is a protein phosphatase inhibitor.

6. The method of claim 5 wherein the protein phosphatase inhibitors is okadaic acid or a salt thereof.

7. The method of claim 6 wherein the salt of okadaic acid is potassium salt, sodium salt, or ammonium salt.

8. The method for treating scars according to claim 5, wherein the protein phosphatase inhibitors change scar cell permeability.

9. The method for treating scars according to claim 8 wherein the protein phosphatase inhibitor inhibits protein phosphatase 2A (PP2A) and is: calyculin A; endothall; fostriecin; microcystin-LF; microcystin-LW; microcystin-RR; α-naphthyl acid phosphate; protein phosphatase 2A inhibitor I₁^{PP2A}, or protein phosphatase 2A inhibitor I₂^{PP2A}.

10. The method for treating scars according to claim 1, wherein the blistering agent is obtained from any natural biological substance.

11. The method for treating scars according to claim 1 wherein administering comprises applying the blistering agent topically.

12. The method of claim 11 wherein the pharmaceutically acceptable carrier is water, alcohol, acetone, an ointment, gel spray, or paste.

13. The method of claim 1 wherein administering comprises injecting the blistering agent intralesionally.

14. The method of claim 12 wherein the pharmaceutically acceptable carrier is saline, water, alcohol, acetone, or oil.

15. The method of claim 1 further comprising causing blistering and necrosis of the scar.

16. The method of claim 15 further comprising forming a scar wound by removing the scar.

17. The method of claim 16 further comprising treating the scar wound.

18. The method of claim 17 wherein the treatment does not cause scarring.
